(19) United States
(12) Patent Application Publication
(10) Pub. No.: US 2020/0038591 A1
(43) Pub. Date: Feb. 6, 2020

(54) PREFILLED AND PRELOADED SYRINGE INJECTION INDUCER

(71) Applicant: Chen TAI, Taoyuan City (TW)

(72) Inventor: Chen TAI, Taoyuan City (TW)

(21) Appl. No.: 16/480,022

(22) PCT Filed: Apr. 28, 2017
(86) PCT No.: PCT/CN2017/082540

§ 371 Date: Jul. 23, 2019

(51) Int. Cl. A61M 5/31

(52) U.S. Cl. CPC A61M 5/31; A61M 2005/323; A61M 2005/3107; A61M 2005/312

(57) ABSTRACT

The invention relates to an injection inducer device for use with a conventional injection syringe prefilled with a liquid medicament or preloaded with a soft capsule containing a liquid medicament. The device includes a seal preloaded in a front adapter end of the injection at position close to an injection opening of the adapter end and formed with a recessed guide groove. The device further includes a removable needle mounted at its front end with an adaptive cover, so that the removable needle can penetrate through the seal along the guide groove to enter a storage chamber of the injection syringe. The seal has a length in the longitudinal direction greater than the bevel length of the removable needle. The invention constitutes a substitution for the conventional medication bottles by virtue of the advantages of high portability, light weight, low frangibility, and high endurance for long distance transport.

### BACKGROUND OF THE INVENTION

### 1. Field of the Invention

The present invention relates generally to a technique regarding a preloaded injection syringe and, more particularly, to an injection syringe device preloaded with a soft capsule containing a liquid medicament, which enables the injection syringe to function as a means of storing medicaments and therefore a substitution of bottle usage. The invention further permits great precision for injection dosage and facilitates portable convenience.

### 2. Description of Related Art

In modern medicine, treatment of a disease by injecting liquid medicaments from soft capsules to the patient has already become a common practice. Hence, vacuum sealed medication bottles are widely used for storage of the soft capsules and medicaments that need be isolated from ambient air, In this case, the soft capsules may be extracted from the medication bottle by inserting a conventional injection syringe into the bottle and drawing the soft capsules into the syringe before an injection treatment.

However, the medical fluid bottles produced in many countries around the world are mostly glass bottles provided with metallic screw caps. The glass bottles inherited the following deficiencies: low endurance for long distance transport, higher rate of breakability, heavy weight, and high occurrence of fluid leakage and microbial contamination due to the loose connection between the bottle and the cap. Furthermore, as each bottle comes with substantially the same dimension and configuration, there poses a higher risk of injection error and lack of precise measurement for dosage control.

In addition, the medication bottles known in the art are likely to pollute the environment after use. Therefore, there is a need in the art for enabling an injection syringe to be pre-filled or pre-loaded with a soft capsule and allowing the injection procedure to be carried out by forward compression of a plunger against the soft capsule stored within the barrel of the syringe, so that the soft capsule is penetrated to release the contents. Such technique would allow greater convenience for each injection and eliminate the drawbacks occurring in the conventional injection procedures which involve the use of medication bottles.

### SUMMARY OF THE INVENTION

A primary object of the invention is to provide an injection inducer device for an injection syringe prefilled with a liquid medicament or preloaded with a soft capsule containing a liquid medicament, which effectively overcomes the drawbacks occurring in the art as described above.

For achieving the object above, the invention provides an injection inducer device for use with a conventional injection syringe. The device comprises a sterile seal preloaded in a front adapter end of the injection at position close to an injection opening of the adapter end, wherein the sterile seal is formed with a recessed guide groove; a removable needle mounted at its front end with an adaptive cover for sleeving upon the adapter end and formed at its front end with a longitudinal bevel, so that the removable needle can penetrate through the sterile seal along the guide groove to enter a storage chamber of the injection syringe, wherein the sterile seal has a thickness in a longitudinal direction greater than a bevel length of the removable needle.

In a preferred embodiment, the device further comprises a plunger formed on its front face with a hole registering with the removable needle, so that the removable needle can insert into the hole after penetrating through the sterile seal and entering the storage chamber of the injection syringe.

For achieving the object above, the invention further provides an injection inducer device for use with a conventional injection syringe and a plunger, where the plunger is sleeved within a storage chamber of the injection syringe and configured to be movable reciprocatingly. The device comprises a spiked disc disposed at bottom of the storage chamber of the injection syringe and mounted centrally with an inner needle which is formed with a longitudinal bevel; and an elongated hole formed at a front end of the plunger and registering with the inner needle of the spiked disc.

For achieving the object above, the invention further provides an injection inducer device for use with a conventional injection syringe and a plunger, where the plunger is sleeved within a storage chamber of the injection syringe and configured to be movable reciprocatingly. The device comprises a spiked disc disposed at bottom of the storage chamber of the injection syringe and formed with a plurality of sharp spikes; and a plurality of sharp barbs disposed on a front face of the plunger, wherein the barbs and the spikes are arranged offset from one other in the longitudinal direction.

For achieving the object above, the invention further provides an injection inducer device for use with a conventional injection syringe. The injection inducer device comprises a spiked disc disposed at the bottom of the storage chamber of the injection syringe and formed with a spiral elastic spike.

For achieving the object above, the invention provides another injection inducer device adapted for receiving a soft capsule. The device comprises an injection syringe mounted at its front end with a first needle and defining a storage chamber, wherein the storage chamber is formed centrally on its bottom face at a junction connecting the storage chamber to the first needle with needle exits in fluid communication with the first needle; and a plunger sleeved within the storage chamber of the injection syringe and adapted to move reciprocatingly, wherein the plunger is mounted at its front face with a plurality of sharp barbs.

In a preferred embodiment, the storage chamber of the injection syringe is formed at its bottom face with a plurality of sharp spikes, wherein the sharp spikes and the sharp barbs are arranged offset from one other in the longitudinal direction.

For achieving the object above, the invention provides another injection inducer device adapted for receiving a soft capsule. The device comprises an injection syringe comprising a front adapter end sealed with a rubber seal, and a storage chamber disposed rear of the adapter end and adapted to receive a soft capsule; a removable needle, mounted at its front end with an adaptive cover adapted to sleeve upon the adapter end, so that the removable needle sleeved within the adaptive cover may be inserted into the storage chamber through the rubber seal, wherein the removable needle is formed at its front end with a longitudinal bevel; and a plunger, sleeved within the storage chamber of the injection syringe and configured to be movable reciprocatingly, wherein the plunger is formed at its front end with an elongated hole registering with the removable needle.

In a preferred embodiment, the longitudinal bevel of the removable needle has a length smaller than the length of the rubber seal, and the length of the longitudinal bevel is smaller than the depth of the elongated hole.

### BRIEF DESCRIPTION OF THE DRAWINGS

FIG. 1a-FIG. 1b are perspective exploded structural view of the injection inducer device according to the first preferred embodiment of the invention;

FIG. 1c-FIG. 1f show structural variations of the sterile seal according to the first preferred embodiment of the invention;

FIG. 2 is a perspective exploded structural view of the injection inducer device according to the second preferred embodiment of the invention;

FIG. 3A is a perspective exploded structural view of the injection inducer device according to the third preferred embodiment of the invention;

FIG. 3B is another perspective exploded structural view of the injection inducer device according to the third preferred embodiment of the invention;

FIG. 4 is a perspective exploded structural view of the injection inducer device according to the fourth preferred embodiment of the invention;

FIG. 5 is a perspective exploded structural view of the injection inducer device according to the fifth preferred embodiment of the invention;

FIG. 6A is a perspective exploded structural view of the injection inducer device according to the sixth preferred embodiment of the invention;

FIG. 6B is another perspective exploded structural view of the injection inducer device according to the sixth preferred embodiment of the invention;

FIG. 7 is a perspective exploded structural view of the injection inducer device according to the seventh preferred embodiment of the invention; and

FIG. 8 is a perspective exploded structural view of the injection inducer device according to the eighth preferred embodiment of the invention.

### DETAILED DESCRIPTION OF THE INVENTION

The above and other objects, features and effects of the invention will become apparent with reference to the following description of the preferred embodiments.

The following embodiments are described in conjunction with the accompanying drawings. It should be noted that the drawings are simplified schematic diagrams illustrating the basic concept of the invention. Therefore, the quantities, configurations and scales of the constituting elements as shown in the drawings are depicted for illustrative purposes only, and it is not meant to limit or otherwise narrow the scope of the invention. The shapes, quantities and scales of the constituting elements in actual implementation may be changed and modified based on the actual need.

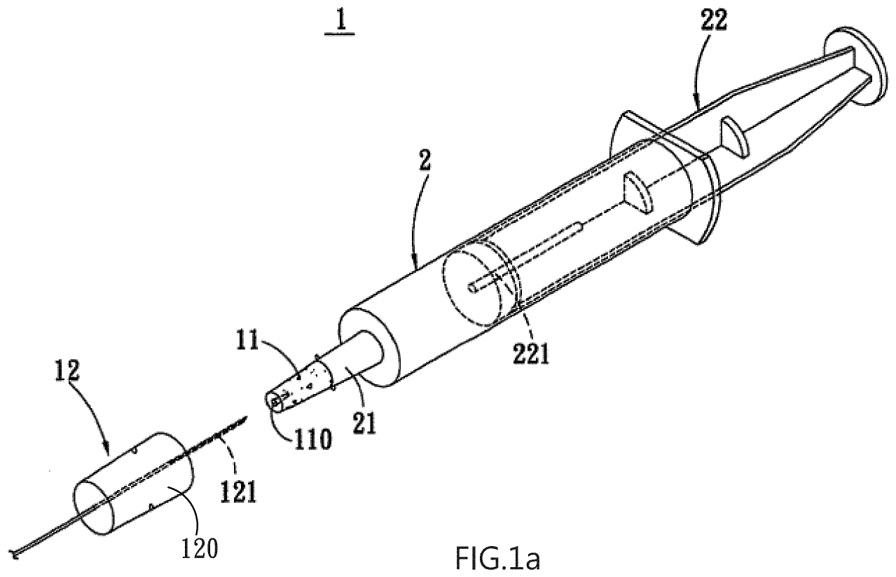

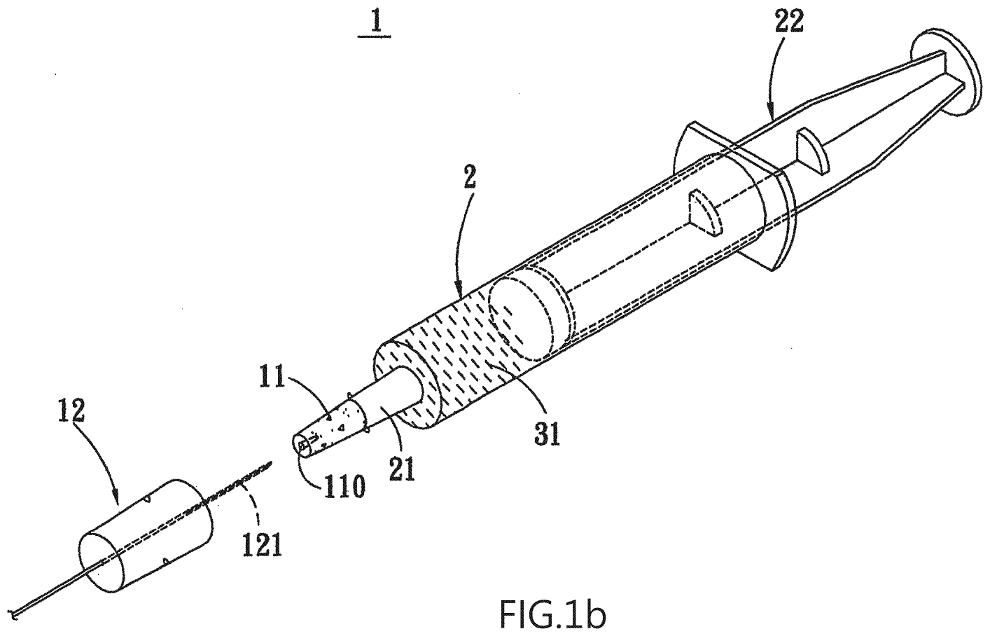

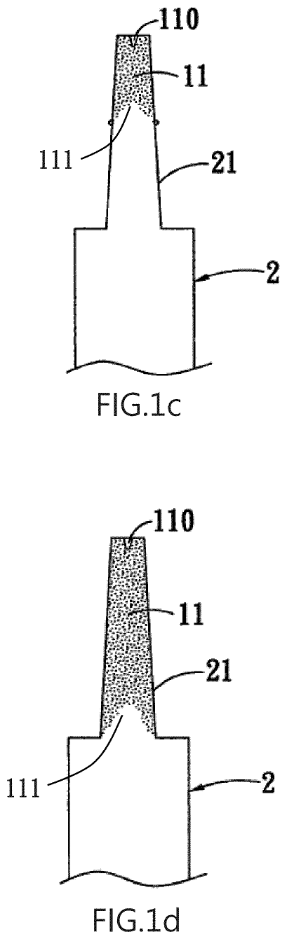

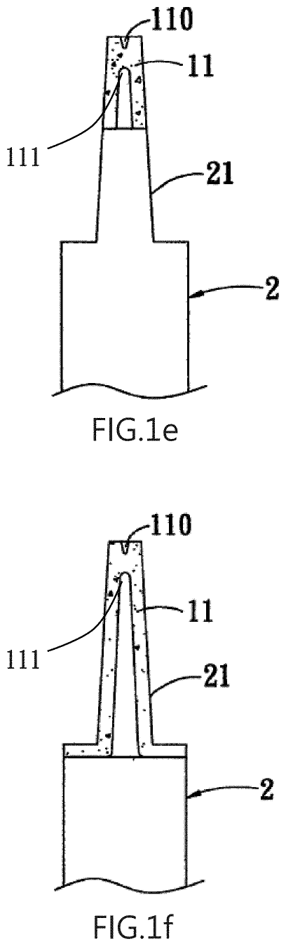

Firstly, FIGS. 1a-1b are the perspective exploded structural diagram according to the first preferred embodiment of the invention, and FIGS. 1c-1f show various configurations of the sterile seal according to the first preferred embodiment of the invention. As shown, the injection inducer device 1 according to the invention is adapted for use with a conventional injection syringe 2 and comprises a sterile seal 11 and a removable needle 12. The sterile seal 11 is preloaded at a front adapter end 21 of the injection syringe 2. The sterile seal 11 is disposed near the injection opening of the adapter end 21 and formed with a recessed guide groove 110. The removable needle 12 is mounted at its front end with an adaptive cover 120 adapted to sleeve upon the adapter end 21. The removable needle 12 is formed at its front end with a longitudinal bevel 121, so that the removable needle 12 can penetrate through the sterile seal 11 along the guide groove 110 to enter the storage chamber of the injection syringe 21. The sterile seal 11 has a thickness in the longitudinal direction, which is smaller than the length of the bevel 121 of the removable needle 12.

In a preferred embodiment, the device further comprises a plunger 22 which is formed on its front face with a hole 221 registering with the removable needle 12. The removable needle 12 is configured so that it can insert into the hole 221 after penetrating through the sterile seal 11 and entering the storage chamber of the injection syringe 2.

FIG. 1c is a schematic cross-sectional view of the sterile seal 11 according to the first embodiment. As shown, the sterile seal 11 is provided at its front end with a guide groove 110, along which the removable needle 12 may be guided to penetrate through the sterile seal 11 and get into the storage chamber of the injection syringe 2 in an accurate manner FIG. 1d to FIG. 1f are schematic diagrams showing various embodiments of the sterile seal 11, all characterized in that the sterile seal 11 has a thickness in the longitudinal direction which is greater than the length of the bevel 121 of the removable needle 12.

In a practical application shown in FIG. 1a, the storage chamber of the injection syringe 2 is provided with at least one soft capsule containing medicaments. As further shown in FIG. 1b, the removable needle 12 is configured to have a shorter bevel length as compared with the length of the adapter end 21, so that the removable needle 12 will not abut against the plunger 22 upon penetrating through the sterile seal 11. The liquid medicament 31 may be preloaded in the storage chamber of the injection syringe 2, while the sterile seal 11 ensure isolation of the soft capsule or liquid medicament 31 stored within the storage chamber of the injection syringe 2 from the ambient. As entering the adapter end of the injection syringe 2, the removable needle 12 is guided by the guide groove 110 of the sterile seal 11, so that it may penetrate through the sterile seal 11 and enter the storage chamber of injection syringe 2 in a straightforward and accurate manner As a result, the liquid medicament 31 stored in the storage chamber is released and flows to the bevel 121 of the removable needle 12, from which it is then delivered to a patient via injection.

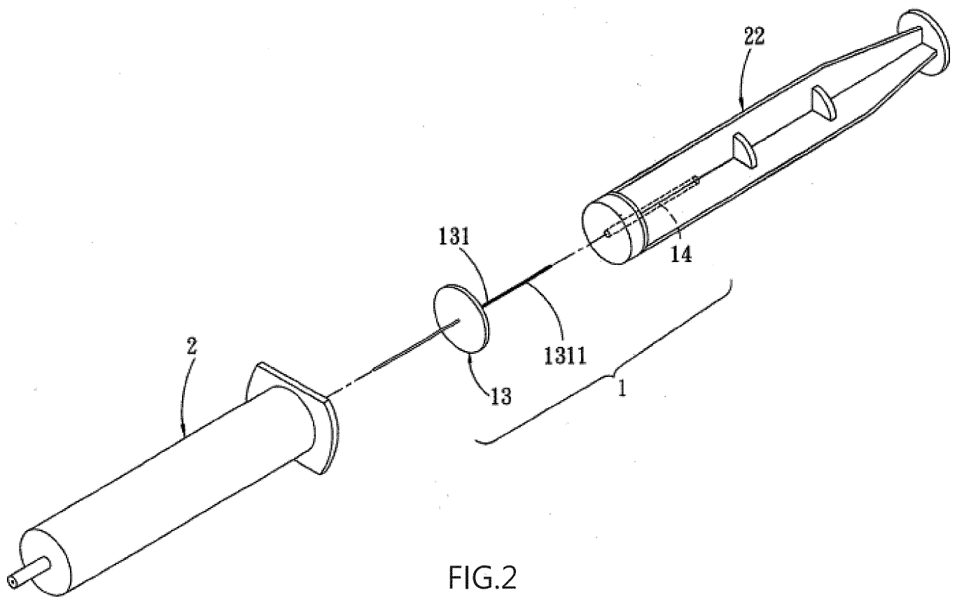

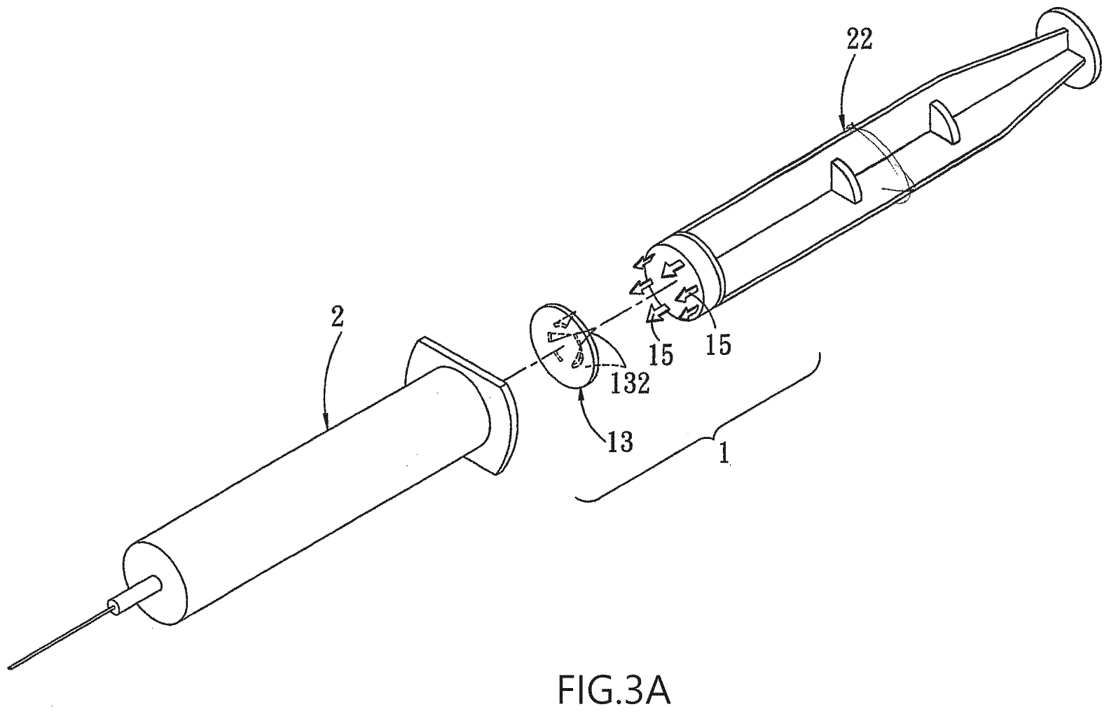

FIG. 2 is a perspective exploded view of the injection inducer device according to the second preferred embodiment of the invention. As shown, the injection inducer device 1 disclosed herein is adapted for use with a conventional injection syringe 2, where a plunger 22 is sleeved within the storage chamber of the injection syringe 2 and configured to be movable reciprocatingly. The injection inducer device 1 comprises a spiked disc 13 and an elongated hole 14. The spiked disc 13 is disposed at the bottom of the storage chamber of the injection syringe 2. The spiked disc 13 is mounted centrally with an inner needle 131 which is formed with a longitudinal bevel 1311. The elongated hole 14 is formed at the front end of the plunger 22 and registers with the inner needle 131 of the spiked disc 13.

In a practical application where a soft capsule (not shown) is preloaded in the storage chamber of the injection syringe 2, by virtue of the structural arrangement described above, when the plunger 22 is sleeved within the storage chamber of the injection syringe 2 and pushed to move forward, the soft capsule is pushed by the plunger 22 to contact the inner needle 131 of the spiked disc 13. As a result, the soft capsule is punctured by the inner needle 131, and the liquid medicament stored in the soft capsule is released and flows through the longitudinal bevel 1311 of the inner needle 131 to the needle of the injection syringe 2, from which the liquid medicament is then administered to a patient via injection. Furthermore, as the plunger 22 is pushed to the bottom of the storage chamber, the inner needle 131 of the spiked disc 13 is inserted into and accommodated perfectly within the elongated hole 14 formed at the front end of the plunger 22.

In addition to the common needle configuration as described above, the inner needle 131 of the spiked disc 13 may be configured to have a bullet-shaped needle tip, so that the inner needle 131 of the spiked disc 13 is adapted to penetrate through the soft capsule straightly.

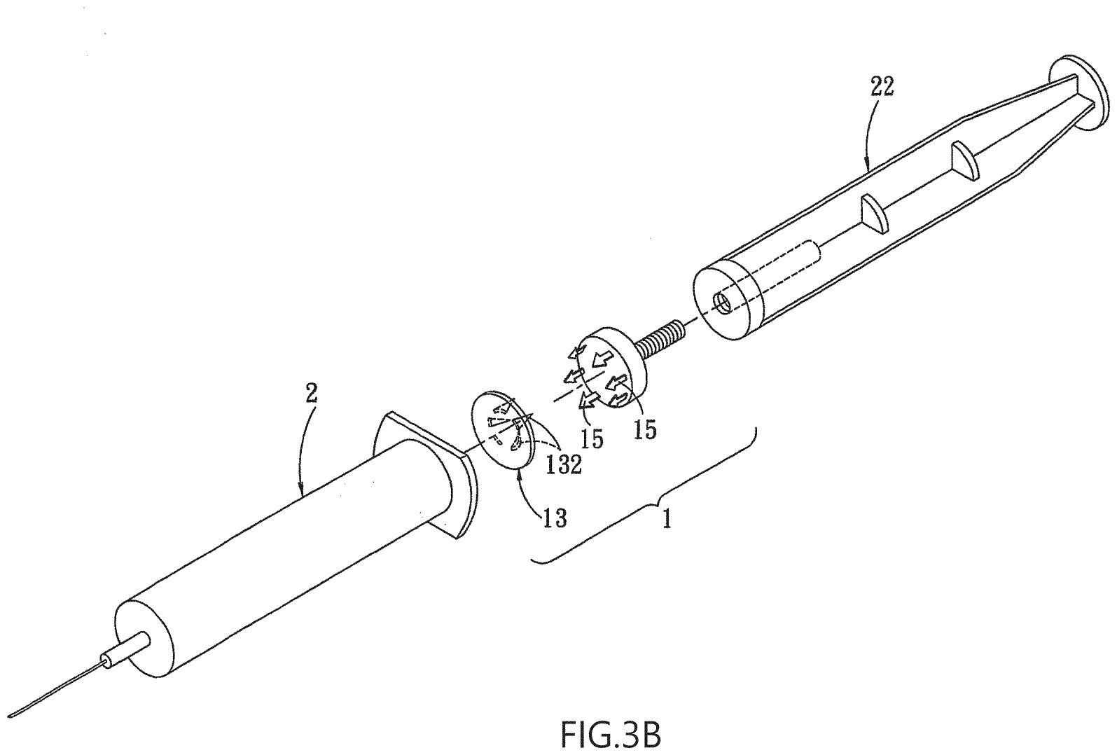

FIG. 3A is a perspective exploded view of the injection inducer device according to the third preferred embodiment of the invention. As shown, the injection inducer device 1 according to the third embodiment of the invention is adapted for use with a conventional injection syringe 2, where a plunger 22 is sleeved within the storage chamber of the injection syringe 2 and configured to be movable reciprocatingly. The injection inducer device 1 comprises a spiked disc 13 and a plurality of sharp barbs 15. The spiked disc 13 is disposed at the bottom of the storage chamber of the injection syringe 2 and formed with a plurality of sharp spikes 132. The sharp barbs 15 are disposed on the front face of the plunger 22, and the barbs 15 and the spikes 132 are arranged offset from one other in the longitudinal direction.

In a practical application where a soft capsule (not shown) is preloaded in the storage chamber of the injection syringe 2, by virtue of the structural arrangement described above, when the plunger 22 is sleeved within the storage chamber of the injection syringe 2 and pushed to move forward, the soft capsule is pushed by the plunger 22 to move toward the spiked disc 13. As a result, the sharp spikes 132 breaks the soft capsule, and the liquid medicament in the soft capsule is released and flows into the needle of the injection syringe, from which the liquid medicament is then administered to a patient via injection. Furthermore, when the plunger 22 is pushed to the bottom of the storage chamber, the sharp barbs 15 may engage the used soft capsule and draw it out of the storage chamber of the injection syringe 2, thereby facilitating the convenience in discarding the used soft capsule.

As shown in FIG. 3B, the sharp barbs 15 may be configured to be a detachable part of the plunger 22.

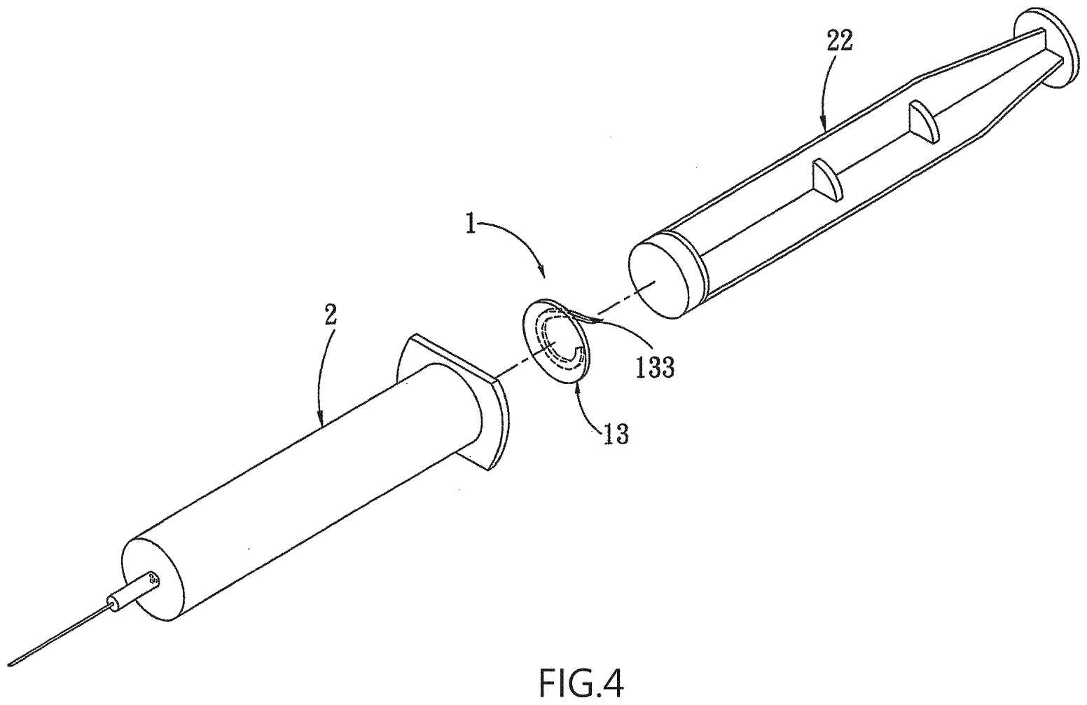

FIG. 4 is a perspective exploded view of the injection inducer device according to the fourth preferred embodiment of the invention. As shown, the injection inducer device 1 according to the fourth embodiment of the invention is adapted for use with a conventional injection syringe 2. The injection inducer device 1 comprises a spiked disc 13 disposed at the bottom of the storage chamber of the injection syringe 2 and formed with a spiral elastic spike 133.

In a practical application where a soft capsule (not shown) is preloaded in the storage chamber of the injection syringe 2, by virtue of the structural arrangement described above, when the plunger 22 is sleeved within the storage chamber of the injection syringe 2 and pushed to move forward, the soft capsule is pushed by the plunger 22 to move toward the spiked disc 13. As a result, the spiral elastic spike 133 breaks the soft capsule, and the liquid medicament stored in the soft capsule is released and flows into the needle of the injection syringe, from which the liquid medicament is then administered to a patient via injection.

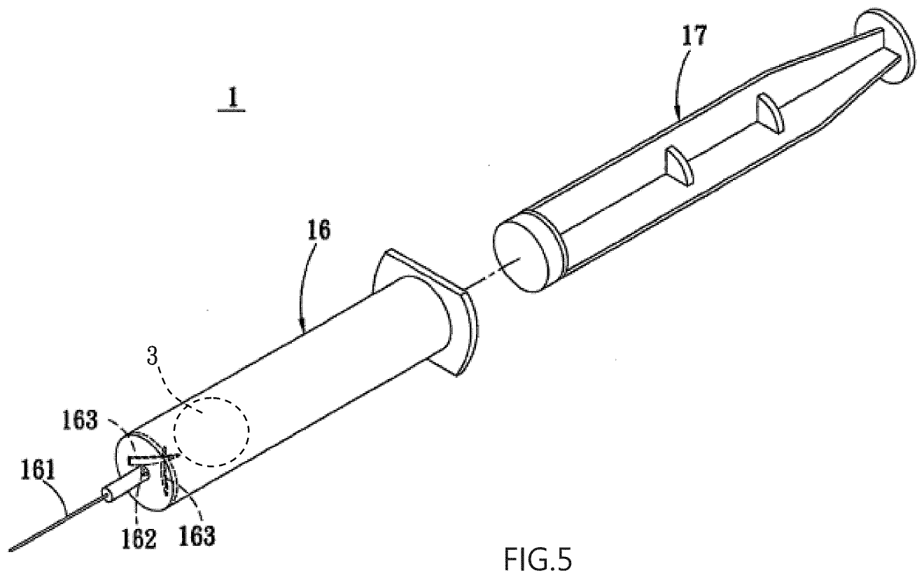

FIG. 5 is a perspective exploded view of the injection inducer device according to the fifth preferred embodiment of the invention. As shown, the injection inducer device 1 disclosed herein is adapted for receiving a soft capsule and comprises an injection syringe 16 and a plunger 17. The injection syringe 16 is mounted at its front end with a first needle 161. A storage chamber is defined by the injection syringe 16 and mounted at its bottom portion with a flexible spike 163 and further formed at its foremost end with needle exits 162 in fluid communication with the first needle 161. The plunger 17 is sleeved within the storage chamber of the injection syringe 16 and adapted to move reciprocatingly.

In a preferred embodiment, the flexible spike 163 is configured in the form of two rectangular-shaped flexible spikes 163 disposed on the bottom surface of the storage chamber. The flexible spike 163 has a length smaller than the radius of the bottom surface of the storage chamber, so that the two flexible spikes 163, upon being pressed, may be flattened on the bottom surface of the storage chamber. In other embodiments, the flexible spike 163 is configured in the form of a spiral-shaped flexible spike 163. It is apparent to those skilled in the art that the amount of the flexible spikes 163 is not limited to two and may be one or three or more.

In a practical application where a soft capsule (not shown) is preloaded in the storage chamber of the injection syringe 16, by virtue of the structural arrangement described above, when the plunger 17 is sleeved within the storage chamber of the injection syringe 16 and pushed to move forward, the soft capsule is pushed by the plunger 17 to move forward. As a result, the flexible spikes 163 break the soft capsule, and the liquid medicament stored in the soft capsule is released and flows through the needle exits 162 to the first needle 161, from which the liquid medicament is then administered to a patient via injection.

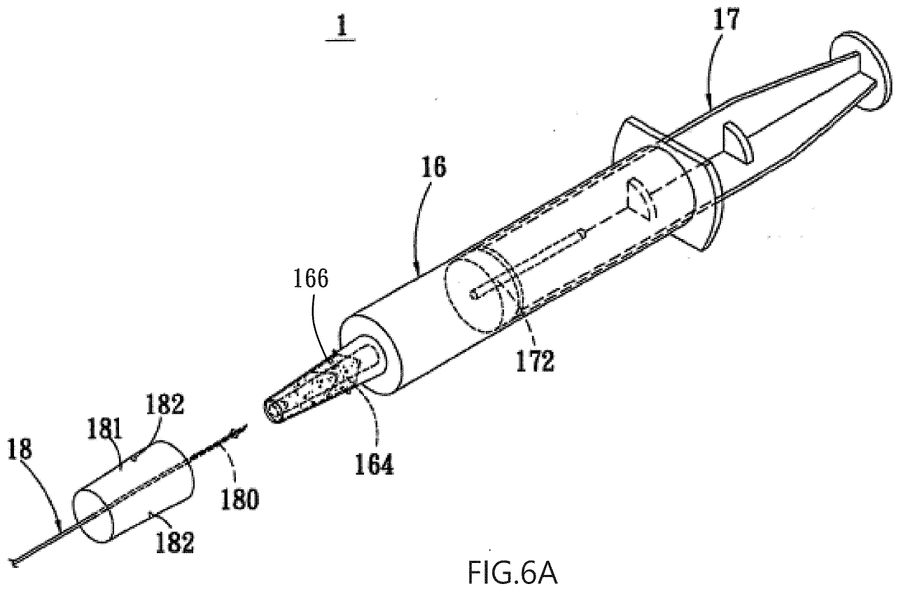

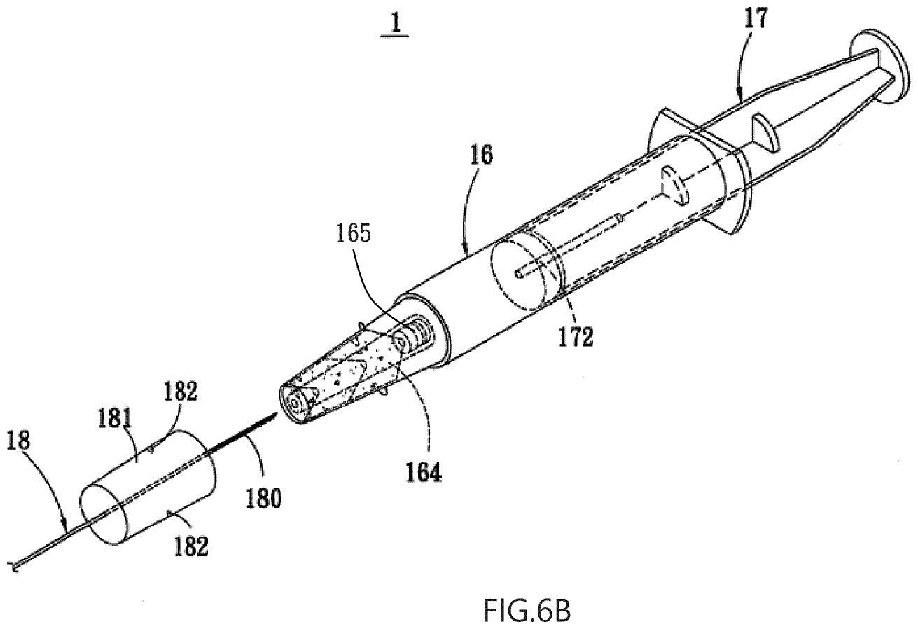

FIG. 6A is a perspective exploded view of the injection inducer device according to the sixth preferred embodiment of the invention. As shown, the injection inducer device 1 disclosed herein is adapted for receiving a soft capsule and comprises an injection syringe 16, a plunger 17 and a removable needle 18. The injection syringe 16 includes a front adapter end sealed with a rubber seal 164, and a storage chamber disposed rear of the adapter end and adapted to receive a soft capsule. The removable needle 18 is mounted at its front end with an adaptive cover 181 adapted to sleeve upon the adapter end of the injection syringe 16, so that the removable needle 18 sleeved within the adaptive cover 181 may be inserted into the storage chamber through the rubber seal 164. The removable needle 18 is formed at its front end with a longitudinal bevel 180. The plunger 17 is sleeved within the storage chamber of the injection syringe 16 and configured to be movable reciprocatingly. The plunger 17 is formed at its front end with an elongated hole 172 registering with the removable needle 18.

In the sixth embodiment, the longitudinal bevel 180 of the removable needle 18 has a length smaller than the length of the rubber seal 164, while the length of the longitudinal bevel 180 is smaller than the length of the elongated hole 172. The adaptive cover 181 is mounted inside with positioning bosses 182, and the adapter end of the injection syringe 16 is formed with protrusions fitting with the positioning bosses 182, so that the adaptive cover 181 is secured on the adapter end of the injection syringe 14. Furthermore, the rubber seal 164 may be configured to consist of multiple tapered sections for rectifying the penetration of the removable needle 18, allowing the removable needle 18 to enter the injection syringe 14 in an accurate manner

In a practical application where a soft capsule (not shown) is preloaded in the storage chamber of the injection syringe 16, by virtue of the structural arrangement described above, when the plunger 17 is sleeved within the storage chamber of the injection syringe 16 and pushed to move forward, the soft capsule is pushed forward by the plunger 17. As a result, the soft capsule is punctured by the removable needle 18 which protrudes into the storage chamber, and the liquid medicament stored in the soft capsule is released and flows through the longitudinal bevel 180 to the tip of the removable needle 18, from which the liquid medicament is then administered to a patient via injection. Furthermore, as the plunger 18 is pushed to the bottom of the storage chamber, the removable needle 18 is inserted into and accommodated perfectly within the elongated hole 18 formed in the plunger 17.

In addition, FIG. 6B is another perspective exploded view of the injection inducer device according to the sixth preferred embodiment of the invention. As shown, the rubber seal 164 may be disposed outside of the adapter end of a conventional injection syringe, and this embodiment can be used in the same manner to achieve the same results as described above.

In addition, the removable needle 18 is configured to have a tapered tip with an apex arranged coaxial with the longitudinal axis of the removable needle 18, so as to prevent an offset during the penetration. The rubber seal 164 is configured to consist of multiple tapered sections, allowing the removable needle 18 to get into the storage chamber accurately and break the soft capsule successfully.

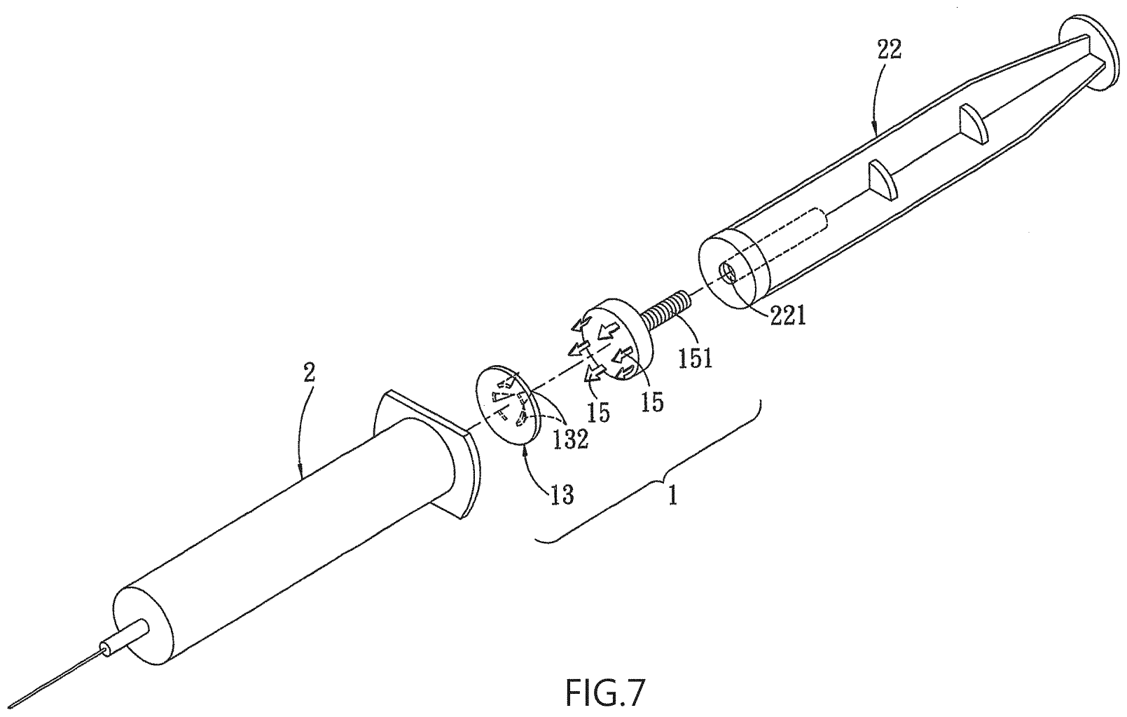

FIG. 7 is a perspective exploded view of the injection inducer device according to the seventh preferred embodiment of the invention. As shown, the injection inducer device 1 disclosed herein is adapted for use with a conventional injection syringe 2, where a plunger 22 is sleeved within the storage chamber of the injection syringe 2 and configured to be movable reciprocatingly. The injection inducer device 1 comprises a spiked disc 13 and a plurality of sharp barbs 15. In this embodiment, the sharp barbs 15 and the plunger 22 are configured as separate parts, with the sharp barbs 15 being integrated into a single-piece configuration provided with a bolt 151 protruding rearward, and the plunger 22 being formed on its front face with a threaded hole 221, thereby allowing the sharp barbs 15 to be directly fastened onto the front face of the plunger 22.

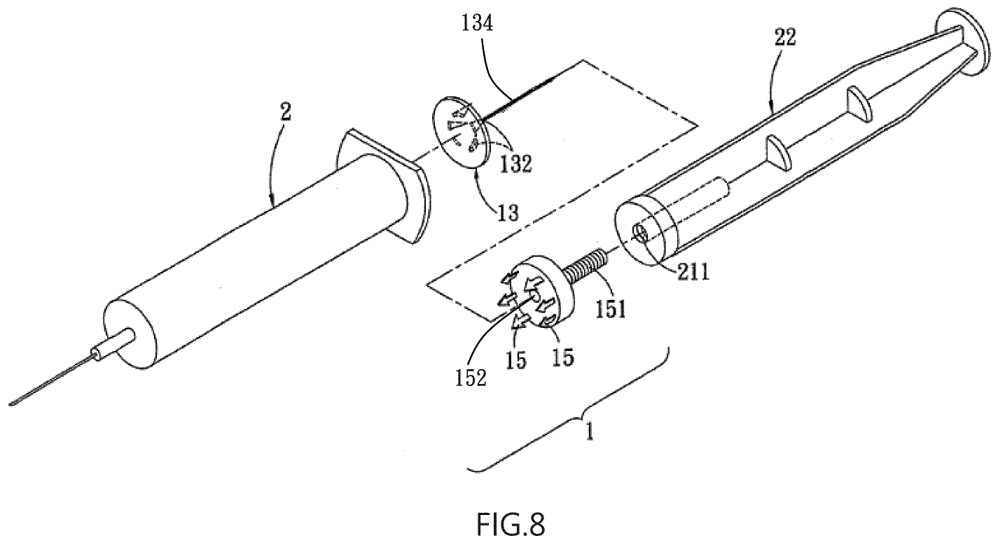

FIG. 8 is a perspective exploded view of the injection inducer device according to the eighth preferred embodiment of the invention. As shown, the injection inducer device 1 disclosed herein is adapted for use with a conventional injection syringe 2 and a plunger 22. Similar to the seventh embodiment, the injection inducer device 1 comprises a spiked disc 13 and a plurality of sharp barbs 15. This embodiment differs from the seventh embodiment in the addition of a second needle disposed centrally with respect to the spiked disc 13, and a perforation formed centrally on the sharp barbs 15 and registering with the second needle, so that the second needle is inserted into the perforation after penetrating through the soft capsule.

With reference to the embodiments described above, a skilled artisan would appreciate that the spiked disc 13, the spiral elastic spike 133, the sharp spikes 132, the sharp barbs 15, the flexible spike 163, and the elongated hole 171 of the plunger 17 are not limited to the configurations described in the embodiments above and may be modified and combined depending on the actual needs.

In contrast to the prior art, the injection inducer device disclosed herein is provided beforehand with a sterile seal in the adapter end of a conventional injection syringe, allowing a liquid medicament or a soft capsule containing the liquid medicament to be preloaded in the storage chamber of the injection syringe. By having the removable needle penetrate the sterile seal, the medicament can be delivered to a patient by injection. It is critical that the thickness of the sterile seal shall be shorter than the bevel length of the removable needle. The conventional medication bottles may be replaced with the invention disclosed herein, in a bid to avoid medication error or difficult dosage management, thus enhancing the convenience and safety in medication.

In addition, the injection inducer device disclosed herein involves provision of a spiked disc in a conventional injection syringe, and one or more soft capsules containing the medicament are preloaded in the storage chamber of the injection syringe. By pushing the plunger, the soft capsules will be collapsed quickly, thereby releasing the medicament stored therein for injection delivery. As such, the invention constitutes an excellent substitution for the conventional medication bottles by virtue of the advantages of high portability, light weight, low frangibility, and high endurance for long distance transport. In addition, the spiked disc described herein may be used in the injection syringe either alone or in combination with a needle in the case where the plunger is formed with an elongated hole registering with the needle. The spiked disc may be configured in various forms, such as sharp spikes, flexible spikes and spiral elastic spikes. It is apparent to those skilled in the art that any structural arrangement capable to penetrating through the soft capsule may be incorporated into the spiked disc, as long as the medicament stored in the soft capsule can be released for injection delivery.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An injection inducer device for use with a conventional injection syringe extending in a longitudinal direction, comprising:
a seal, preloaded in a front adapter end of the injection syringe at position close to an injection opening of the adapter end, wherein the seal is formed with a recessed guide groove;
a removable needle, mounted at its front end with an adaptive cover for sleeving upon the adapter end and formed at its front end with a longitudinal bevel which has a length in the longitudinal direction, so that the removable needle can penetrate through the seal along the guide groove to enter a storage chamber of the injection syringe, wherein the seal has a length in the longitudinal direction greater than the length of the bevel of the removable needle.

2. The injection inducer device according to claim 1, further comprising a plunger formed on its front face with a hole registering with the removable needle, so that the removable needle can insert into the hole after penetrating through the sterile seal and entering the storage chamber of the injection syringe.

3. An injection inducer device comprising:
an injection syringe, extending in a longitudinal direction, in which a storage chamber is defined;
a plunger, sleeved within the storage chamber of the injection syringe and configured to be movable reciprocatingly; and
a spiked disc, disposed at bottom of the storage chamber of the injection syringe.

4. (canceled)

5. An injection inducer device adapted for receiving a soft capsule, comprising:
an injection syringe, mounted at its front adapter end with a first needle and defining a storage chamber, wherein the storage chamber is formed centrally on its bottom face at a junction connecting the storage chamber to the first needle with needle exits in fluid communication with the first needle; and
a plunger, sleeved within the storage chamber of the injection syringe and adapted to move reciprocatingly, wherein the plunger is mounted at its front face with a plurality of sharp barbs.

6. The injection inducer device according to claim 5, wherein the storage chamber of the injection syringe is formed at its bottom face with a plurality of sharp spikes, wherein the sharp spikes and the sharp barbs are arranged offset from one other in the longitudinal direction.

7-10. (canceled)

11. The injection inducer device according to claim 1, wherein the storage chamber of the injection syringe is preloaded with a liquid medicament or a soft capsule containing a liquid medicament.

12. The injection inducer device according to claim 2, wherein the seal has a rear end facing the storage chamber of the injection syringe, and the rear end is formed with a longitudinal recess registering with the guide groove and adapted to receive the removable needle.

13. The injection inducer device according to claim 12, wherein the seal is a sterile seal.

14. The injection inducer device according to claim 13, wherein the seal is configured in the form of a rubber seal filled in the adapter end of the injection syringe.

15. The injection inducer device according to claim 14, wherein the adaptive cover is mounted inside with positioning depressions, and the adapter end of the injection syringe is formed with protrusions fitting with the positioning depressions.

16. The injection inducer device according to claim 14, wherein the rubber seal is configured to consist of multiple tapered sections for rectifying penetration of the removable needle.

17. The injection inducer device according to claim 14, wherein the rubber seal is threaded to the adapter end.

18. The injection inducer device according to claim 3, wherein the spiked disc is mounted centrally with an inner needle, and the plunger is formed at its front end with an elongated hole registering with the inner needle.

19. The injection inducer device according to claim 3, wherein the spiked disc is formed with a spiral elastic spike.

20. The injection inducer device according to claim 3, wherein the spiked disc is configured to include at least one flexible spike disposed on a bottom surface of the storage chamber, and the at least one flexible spike has a length smaller than a diameter of the bottom surface of the storage chamber, so that the at least one flexible spike, upon being pressed, may be flattened on the bottom surface of the storage chamber.

21. The injection inducer device according to claim 3, wherein the spiked disc is formed with a plurality of sharp spikes and the plunger is formed on its front face with a plurality of sharp barbs, and wherein the barbs and the spikes are arranged offset from one other in the longitudinal direction.

22. The injection inducer device according to claim 21, wherein the sharp barbs are configured as a detachable part of the plunger.

23. The injection inducer device according to claim 22, wherein the sharp barbs are integrated into a single-piece configuration provided with a bolt protruding rearward, and the plunger is formed on its front face with a threaded hole.

24. The injection inducer device according to claim 21, wherein the spike disc is mounted centrally with a second needle, and the single-piece configuration of the sharp barbs is formed centrally with a perforation registering with the second needle.

25. The injection inducer device according to claim 3, wherein the injection syringe is mounted at its front adapter end with a first needle, and the spiked disc is formed with a needle exit in fluid communication with the first needle.
